(19) United States
(12) Patent Application Publication
(10) Pub. No.: US 2013/0252340 A1
(43) Pub. Date: Sep. 26, 2013

(54) METHOD AND DEVICE FOR TESTING TREATMENTS WHICH INTRODUCE ENERGY INTO OBJECTS

(71) Applicant: Fraunhofer-Gesellschaft Zur Forderung Der Angewand ten Forschung E.V., Munchen (DE)

(72) Inventors: Thomas Haertling, Dresden (DE); Anton Mayer, Gilching (DE); Jörg Opitz, Dresden (DE); Jürgen Schreiber, Dresden (DE); Susan Derenko, Dresden (DE); Christiane Wetzel, Dresden (DE)

(21) Appl. No.: 13/990,652

(22) PCT Filed: Nov. 28, 2011
(86) PCT No.: PCT/DE2011/002080

§ 371 Date: May 30, 2013

(30) Foreign Application Priority Data
Nov. 30, 2010 (DE) 10 2011 053 723.3

(51) Int. Cl. G01N 21/64; A61L 2/28

(52) U.S. Cl. CPC G01N 21/643; A61L 2/28
USPC 436/1; 422/82.08

(57) ABSTRACT

The method comprises the following steps: bonding at least one chemical, optically luminescent compound (3) onto at least one indicator element (6) for testing the treatments (14), wherein at least one luminescence property of the chemical compound (3) can be changed; assigning at least one indicator element (6) to the object (1); wherein the indicator element (6) and the object (1) are simultaneously subjected to the same conditions of the energy-introducing treatment (14); changing the luminescence property of the chemical compound (3), wherein the level of the change to the luminescence property depends upon the energy-introducing treatment (14); irradiating (13) the chemical compound (3) with electromagnetic radiation directed onto the indicator (6) for excitation of the luminescence during the energy-introducing treatment or following the energy-introducing treatment (14).

The invention relates to a method and a device for investigating treatments applying energy to objects. An energy entry into an object is performed during the treatment, wherein the object can be a subject or a material. The energy entry can be performed mechanically, thermally, by irradiation or by electrical and/or magnetic force effects.

Such treatments for the production or for the modification of objects are performed in many areas. Frequently a certain minimum entry of energy is required for this purpose, which minimum entry of energy is decisive for the desired success of the treatment.

Therefore there exists the requirement of checking, if a sufficient energy entry has taken place, wherein the energy entry can he performed possibly free of destruction, with low expenditure, in a short time, automated, and without danger.

However, most of the known testing methods will not fulfill these requirements, at least not sufficiently in the desired volume. The checking with x-rays or with another radiation is either expensive (computer tomography, nuclear or electron spin resonance) or in case of use of dose meters, wherein a very large time volume is required in case of an irradiation in order to be able to perform a sufficiently accurate checking.

A dose meter is described in the U.S. Pat. No. 5,569,927 A, where also an optical fluorescence excitation can be employed. Such a dose meter can be employed for determining the radiation dose of different ionized radiations (for example beta, gamma, and x-ray radiation). A dose meter material mixed with a polymer is here employed, wherein also several chemical compounds are recited, which compounds can be doped.

A device for a destruction free determination of the dose of radiation is described in the United States patent application publication 2004/0159803, wherein a luminescence material is subjected to an ionizing radiation and a luminescent material is thereby formed. The luminescent material is irradiated with a light source for luminescence excitation and the therewith detected luminescent light is detected in order to determine the value of the fluorescent emission which was obtained by the first irradiation.

A device for determining an energy entry by absorption under an irradiation for a sterilization is described in the United States patent application publication 2004/0211916 A. Here the object to be sterilized with a material absorbing the radiation in a quantified amount and a cooling agent employed in a container and then an irradiation is performed. The value of the absorbed energy is determined, which is obtained by a temperature determination.

Thus in particular the sterilization as is required in the medical field, is problematical. Implants, prostheses, medical apparatus and instruments have been sterilized predominantly in autoclaves up to now. Here the reaching and maintaining of a minimum temperature over a sufficiently large time period is required. It is in addition problematical that at least during the withdrawal from the autoclave the sterility can be interfered with.

In order to avoid these disadvantages, a radiation was employed for sterilizing in order to kill germs. The use of an electron irradiation was introduced in the most recent time. Thereby exists the possibly of sterilizing implants, prostheses, medical apparatus and instruments, which are hermetically closed relative to the environment. The respective sterilized implants, prostheses, medical apparatus, and instruments can be maintained sterile in the container over longer time periods and can only shortly before their use be removed from the container.

A certain proof of a performance of a sufficient sterilization can at least not be performed as long as the implants, prostheses, medical apparatus and instruments are still enclosed by the container.

In further different methods, wherein the objects are subjected to a thermal treatment, the proof about the success of the performed treatment can be performed free of destruction only with a substantial expenditure, which is true at least with complex three-dimensional geometries, which is the case for example with undercuts.

It is an object of the invention to furnish a method and a device for testing of objects with respect to energy entering treatments, which are formed such to test objects in energy entering treatments without destruction, wherein the testing is to be performed with low expenditure, in a short period of time, at least with a sufficient proof precision and without danger.

The object is resolved with the features of patent claims 1 and 12.

The method for testing of an object subjected to energy entering treatments exhibits the following steps:
bonding of at least one chemical, optical compound capable of luminescence in/on at least one indicator element for testing the treatments, wherein the luminescence property of the chemical compound is changeable, coordination of at least one indicator element to the object, wherein the indicator element and the object are simultaneously subjected to the same conditions of the energy entering treatment,
changing of the luminescence property of the chemical compound, wherein the level of the change of the luminescence property depends on the energy entering treatment,
irradiation of the chemical compound with an electromagnetic radiation directed toward the indicator element for exciting of luminescence during the energy entering treatment or following to the energy entering treatment for detection of energy entering instantaneous treatment or energy entering performed treatment,
time resolved and/or spectrum resolved detection of the electromagnetic radiation by the indicator element emitted in consequence of luminescence of the chemical compound during the luminescence triggering irradiation or after the switching off of the luminescence triggering the irradiation,
ready making of time resolved and/or spectrum resolved detection signals at at least one evaluation unit, wherein there occur
a comparison between the detection signals and the reference signals from at least one prior determined reference value and/or at least one reference wavelength or from a reference wavelength spectrum in the evaluation unit, and
a detection determination of one treatment performed energy entering on the object, and
at least one indicator in an indicator unit on the basis of the accomplished changed luminescence property of the chemical compound relating to the presence of at least one energy entry into the object.

At least one chemical compound is employed, which exhibits a reversible change of the luminescence property or an irreversible change of the luminescence property, for detection during the energy entering treatment.

For example, the irreversibility of the change of one optical property of the employed chemical compound represents a time stable change of at least one optical property of the chemical compound, which change is caused by the planned energy entry. The change can either concern a shortening or an extending of the luminescence lifetime τ, and change in the luminescence spectrum or the increase or decrease of the luminescence intensity I_{L}. Based on the irreversibility the above recited time stable change can be checked at each time after the energy entering.

The luminescence lifetime τ and/or an associated luminescence intensity can be determined at a pre-given time and can be compared with at least one reference value.

The radiation directed towards the indicator element for the exciting of the luminescence in the chemical compound can be performed as pulses in case of a time resolved detection.

The presence or absence of at least one wavelength in the wavelength spectrum of the radiation emitted following to luminescence can be detected.

Several chemical compounds can be employed at one or several indicator element(s), which change their luminescence properties upon the reaching of different energy entries.

The energy entering treatment(s) of medical implants, prostheses, medical apparatus and instruments with electron irradiation can be performed for their sterilization.

Furthermore, the energy entering treatment and the detection of time resolved and/or spectrum resolved luminescence detection signals can be performed in the detector at objects accepted in hermetically closed containers and/or the indicator element(s).

The chemical compound(s) can be employed as a powder with an average particle size in the region of 0.001 μm to 30 μm.

Furthermore in the equipment of an indicator element, the chemical compound(s) can be received in a separate container or together with a matrix material can be printed on a substrate or on the wall of the container or can be attached immediately at the respective object or can be embedded in a polymer working material or in the work material of the object. At least one indicator element formed in this way can be employed for testing thereby.

Doped zinc sulfate, doped calcium sulfite, doped aluminum gallate, doped calcium tungstate, doped aluminate-chromate, doped rare earth compounds, as for example rare earth fluorides, or doped oxi-sulfides or doped metal oxides can be employed as chemical compounds.

The treatments are furnished in at least one arrangement for performing an energy entering treatment. The device for checking objects of energy entering treatments comprises
at least one indicator element coordinated to an object, which indicator element is formed with at least one chemical compound, which chemical compound is optically capable of luminescence and the luminescence property of which chemical compound is changeable, and
at least one radiation source with an electromagnetic radiation, which as desired emits with the chemical compound, which exhibits the treatment induced obtained change of the luminescence property for exciting of luminescence on the indicator element,
at least one optical detector, which detector is formed for time resolved and/or spectrum resolved detection of luminescence radiation emitted by the chemical compound,
at least one evaluation unit for detection determination of at least one treatment performed energy entering onto the object.

The coordination of at least one indicator element to the object can be performed such that the chemical compound(s) is/are received by a separate container or together with one matrix working material are printed onto a substrate or onto the wall of the container or are embedded immediately at the respective object or in a polymer work material, the work material of the object.

According to the invention method, where an energy entry is obtained by warming and/or irradiation or also by mechanical or other physical forces or energies, the object of the treatment, in particular of a particulate irradiation, is subjected to the corpuscular radiation. Here in addition to electron radiation also neutron or ion radiation can represent corpuscular radiation. An irradiation at a treatment can however also be performed with x-ray radiation, UV radiation, or IR radiation.

It is essential in connection with the invention method that the luminescence properties of the chemical compound during the treatments can reversible or irreversible change. This can be the case upon the reaching or surpassing of a minimum energy entry. Here for example a change of the crystal lattice structure of the respective chemical compound and/or the stoichiometry of the chemical compound and thereby can change the wavelength spectrum of the emitted luminescence radiation. Then at least one other wavelength can be contained in the wavelength spectrum or at least one wavelength cannot be any longer present in the wavelength spectrum.

During or in connection to the treatment, the chemical compound of the indicator element is irradiated with an electromagnetic radiation for exciting the luminescence. The emitted luminescence radiation is detected during the irradiation or between individual pulses of the irradiation or after the switching off of the irradiation and a comparison is performed of the in this way captured measurement signals with at least one previously determined reference value and/or a reference wavelength and or a reference wavelength spectrum is performed during the irradiation or between individual pulses of the irradiation or after the switching off of the irradiation. It can thereby be recognized if a certain energy entry was performed during the treatment or not. Preferably, the intensity of the emitted luminescence radiation is time resolved detected. A pulsed irradiation is only required with a time resolved detection and is to be performed for such a detection.

In case of a non performed or, respectively, not sufficiently performed change of the luminescence properties it can be determined that the treatment was not successful, if for example a certain level or a certain threshold value as a reference value was too low or also was surpassed.

In contrast to this it can however also be checked, if an energy entry has surpassed a value during treatment and thereby the energy entry was too high, such that when an undesired damaging of the respective object occurred during treatment, or at least however with high probability could occur.

The chemical compounds, the luminescence properties of which change reversibly by a treatment, can be employed in the invention method for exciting of luminescence and the detection are performed during the energy entering treatment.

The chemical compounds, the luminescence properties of which change irreversibly by a treatment, can be employed in the invention method such that the irradiation for the excitation of luminescence and the detection are preferably performed following to the energy entering treatment.

It is preferred that during the detection of the luminescence to determine the luminescence lifetime τ, which is specific for a chemical compound and a crystal lattice present, be determined and compared with at least one reference value. The luminescence lifetime τ can be determined from an exponent law in case the luminescence is determined by one or by several relative to time well separated electron transitions or with one potential law in case of relative to time overlapping electron transitions. It is here advantageous that no dependency on an absolute value of the determined luminescence intensity has to be taken into consideration.

The luminescence lifetime τ will change significantly in case of a sufficiently high energy entry during the treatment, since the crystal lattice structure of the respective chemical compound and thereby also the luminescence lifetime τ as a consequence of the energy entry during the treatment has changed irreversibly. This way a safe proof can be recorded about the success of the treatment. The lifetime τ can be determined such that the time from the switching off of the radiation source for the excitation or starting with a maximum of the emitted luminescence radiation intensity up to the reaching of a threshold value during the decay of the emitted luminescence radiation is measured.

A time resolved detection can also be performed such that the intensity of the emitted luminescence radiation at a certain constant time is determined in the case after the switching off or the termination of the irradiation for the luminescence excitation is determined and the intensity is compared with a reference value.

The radiation can be performed with individual pulses, wherein the pulse length can be in the region of 0.1 ms to 100 ms, and preferably are up to 1 ms. For the selection of the pulse length, the energy density in the focal point of the radiation employed for the luminescence excitation and the respective chemical compound to be excited should be considered. A largest number possible of electrons shall be in an excited state. The measurement of the decay of excited states in the luminescence can preferably be started immediately at the end of an individual pulse of the radiation employed for the luminescence excitation. The detection can be performed at this point in time. The detection can then be performed in a preferred wavelength region and therein be performed for at least one wavelength. Such a time measurement window shall be smaller than 5 ms if possible, and preferably smaller than 1 ms if possible . The luminescence intensity can be at least 100-fold, preferably at least 500-fold, more preferably at least 1000-fold be measured within this time measurement window, such that a sufficient scanning rate is achievable.

Since the determination for the excitation and the decay of the luminescence sequence can be performed with several pulses, the precision can be increased by an average formation with the in this way attainable multiple measurements and the signal to noise ratio can be improved.

The electromagnetic radiation is directed in a defined way onto an indicator element for the excitation of luminescence in order to obtain reproducible situations and comparable measurement results. It is advantageous to work with constant intensity and energy. This concerns the individual pulses by way of which the electromagnetic radiation is directed onto an indicator element. Also the energy density in the focal point, which is disposed in the irradiated plane, should be maintained at least nearly constant.

The excitation of the luminescence is performed with electromagnetic radiation of at least one wavelength, wherein the wavelength is particularly preferred disposed in the wavelength region of the infrared light. The one or several wavelength(s) for the excitation shall not coincide with the wavelength(s) of the luminescence radiation. Advantageously the indicator elements can be irradiated with electromagnetic radiation from a wavelength region of the UV light, of the visible light, and/or infrared light or also with X-ray photons. The respective chemical compound is to be selected correspondingly. Preferably a monochromatic electromagnetic radiation with a pre-given wavelength can be employed for the excitation. The selection of the optical detector can be performed with consideration of the wavelength to be detected. Advantageously photodiodes, preferably on a silicon basis, are employed, which are above a wavelength of 1300 nm not or only in a very small measure sensitive. In order to avoid an influencing by electromagnetic radiation with undesired wave lengths, for example the ambient light, an adapted band pass or long pass filter can be disposed in front of an optical detector. In case of wavelengths above 1300 nm to be detected, photodiodes based on germanium can be employed for the detection.

It is in addition advantageous to dispose a collimating optical element into the beam path of the radiation employed for the excitation and/or in front of an optical detector such that the radiation collimates onto an indicator element or impinges the optical detector and thereby a nearly constant energy density can be achieved in the focal point or on the image on the optical detector also in case of different distances between the radiation source for an exciting and the detector for the respective indicator element.

A spectral resolved detection can be performed in addition to a time resolved detection as previously explained or alone. A spectrometer can be employed as an optical detector for this purpose, with which certain wavelengths within the wavelength spectrum of the emitted luminescence radiation can be captured. It can occur through an energy entry at the treatment that one or several wavelength(s) are not any longer present in the wavelength spectrum or at least one wavelength is new in the wavelength spectrum. Also a band pass filter or a cut off filter can be disposed in front of an optical detector for such a determination instead of a spectrometer, with which optical detector a desired and pre-given wavelength selection is achievable during the detection.

Several chemical compounds can be employed with one or several indicator element(s), which chemical compounds change their luminescence properties reversible or irreversible upon reaching of different energy entries according to the possibility of the invention. The safety of the detection over the success of the performed treatment can thereby be further enhanced and in addition a quantification can be achieved. Thus the possibility exists to further perform a detection about which temperature or irradiation dose was in fact achieved or performed at the execution of the treatment.

As was already indicated in the introduction of this description, an electron irradiation of medical implants, prostheses, medical apparatus and instruments can be performed as a treatment for their sterilization.

Not only in this case can it be advantageous to perform the treatment and detection with objects received in hermetically closed containers (packaging) and at the indicator element(s).

The chemical compound(s) can be employed as a powder and can be employed with an average particle size in the region of 0.001 μm to 30 μm.

In an equipment as indicator element, the chemical compounds can be received in a special container (polymer foil bag) or together with a matrix material printed on a substrate or printed on the container wall or the chemical compound(s) is/are immediately attached at the respective object or is/are embedded in a polymer material. A chemical compound however can also be embedded in the material out of which the object was produced or can be embedded in the object material, which is subjected to an energy entering treatment such that an integrated indicator element is present.

At least one indicator element formed in this manner can be employed or laid in a container according to the invention method. For example, a printable ink/paste can be produced, wherein particles of the respective chemical compound is contained. This ink can immediately be printed on the respective object, on a carrier or on a container wall.

At least a part of a container wall can be formed with particles embedded in a polymer. The employed polymer however should at least be sufficiently transparent for the emitted luminescence radiation. Here for example a container can be a blister pack which is in part formed out of such a polymer. Possibilities for an embedding of particles in polymer is a for example a common extrusion.

A relative small part of the chemical compound is required with an ink or embedding in a polymer. Parts below 5 vol.-%, however also smaller than 2%, or even 1% can be sufficient without problem.

Examples for chemical compounds, which can be used in the invention are doped zinc sulfite, doped calcium sulfite, doped aluminum gallate, doped aluminate chromate, doped rare earth compounds, as for example rare earth fluorides, or doped oxi-sulfides, for example NaYF or Y₂O₂S, or also doped metal oxides.

Ag, Au, Cu or also differing rare earth metals, preferably Yb, Er or Tm can be employed for the doping. Also very small parts are sufficient for the doping.

A radiation source and an optical detector can here be received in a common apparatus or housing. Also an electronic evaluation unit and control unit can be integrated therein, which control unit controls the irradiation leading to the excitation of the luminescence and the measurement signals captured with the at least one optical detector can be evaluated with the evaluation unit. Here also can be present a display for displaying a detection result as well as an interface for a data exchange. A manually led and actuated apparatus can be employed, which automatically performs the detection process and which can display immediately the detection result. Reference values, which are specific particularly for not influenced chemical compounds, can be stored in a memory storage, which memory storage can be integrated in the electronic evaluation unit and control unit, wherein a radiation source emitting the excitation radiation is controlled by the control unit, and wherein measurement signals captured by a detector can be evaluated. The reference values and the reference wavelengths can then be used for the detection of the running or finalized performance of the energy entering treatment as already explained.

In particular in cases of applications of the invention at difficult reachable positions, it is advantageous to lead the radiation employed for the excitation and the luminescence radiation emitted by an indicator element through light-wave conductors (optical fibers), which conductors are deformable and flexible as far as possible.

The invention method works without contacts and free of destruction. The method can be performed automatically. An interference of the respective object can at least to a large extent be avoided. If the detection is performed at objects, then it is not necessary to open the container or to destroy the container for the performance of the method. The container has to exhibit here at least one region, which is transparent for the employed radiation and the radiation emitted by the indicator element.

Sterilized medical implants, prostheses, medical apparatus and instruments can be held up to shortly before an immediate use in a packaging hermetically closed and sterile. Here the sterility can be checked and tested also shortly before the opening or, respectively, the usage.

A change of the luminescence properties can also occur by way of a heat treatment, wherein the energy entry is furnished by a heat treatment.

For example the method can be employed for detecting a sufficient performance of a tempering of objects made of glass or ceramics. Here an indicator element can be placed immediately at such object during tempering. Usually this tempering occurs at temperatures in the region between 400 degrees centigrade to 600 degrees centigrade. Rare earth fluoride compounds can be employed as chemical compounds. An indicator element can be produced with a dispersion, which contains such a chemical compound and which exhibits a pre-given viscosity, by simple application or gluing on an object to be tempered. The luminescence lifetime τ of the chemical compound can previously be determined for selected temperatures of a heat treatment or can be known and be used as reference value(s).

After the tempering has been performed, a pulsed irradiation of the indicator element for luminescence excitation can be performed. The luminescence lifetime τ can then be determined with an optical detector by time resolved detection and can be compared with at least one reference value as previously mentioned. This way a detection proof about the success of the performed treatment can be recorded or possibly also an energy entry of too high a value can be shown.

An indicator element can here be applied to an object such that it is not visible or only slightly negatively influences the esthetic impression.

In addition to glass and ceramics, the method can also be employed at malleable cast iron parts or electronic products (for example circuit boards), which were subjected to a heat treatment.

Further formations and other special versions of the method and the device are given in further sub claims.

The method and the device are to be explained in more detail by way of an example in the following. There is shown:

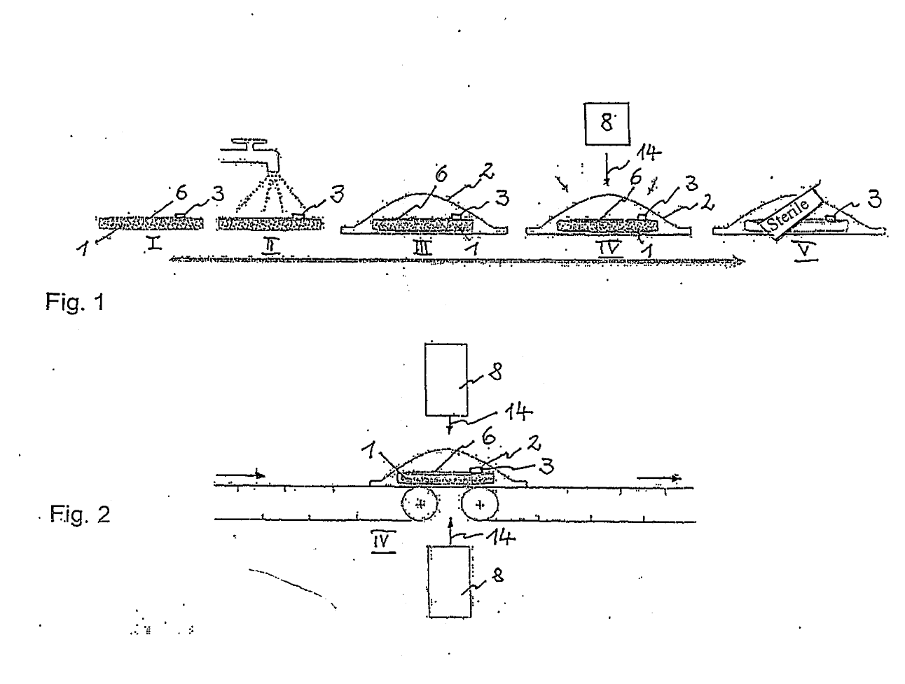

FIG. 1 the procedure up to the sterilization of an object in a container in several steps,

FIG. 2 the procedure during detection of the performed sterilization in a schematic presentation,

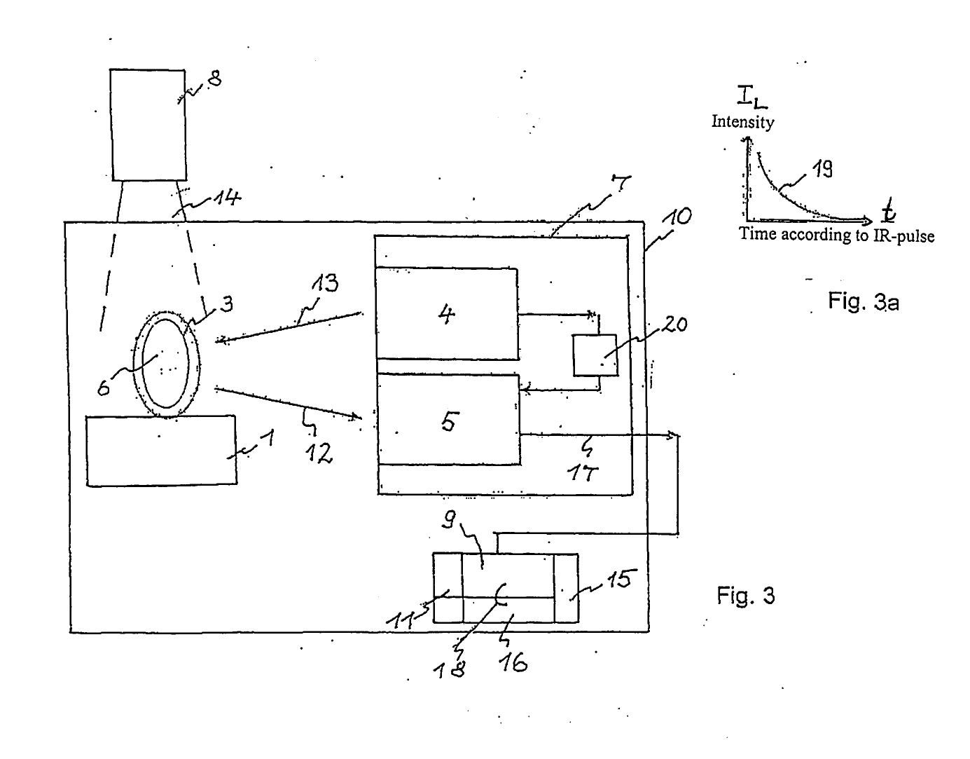

FIG. 3 the procedure at the detection of the performed sterilization in a schematic presentation,

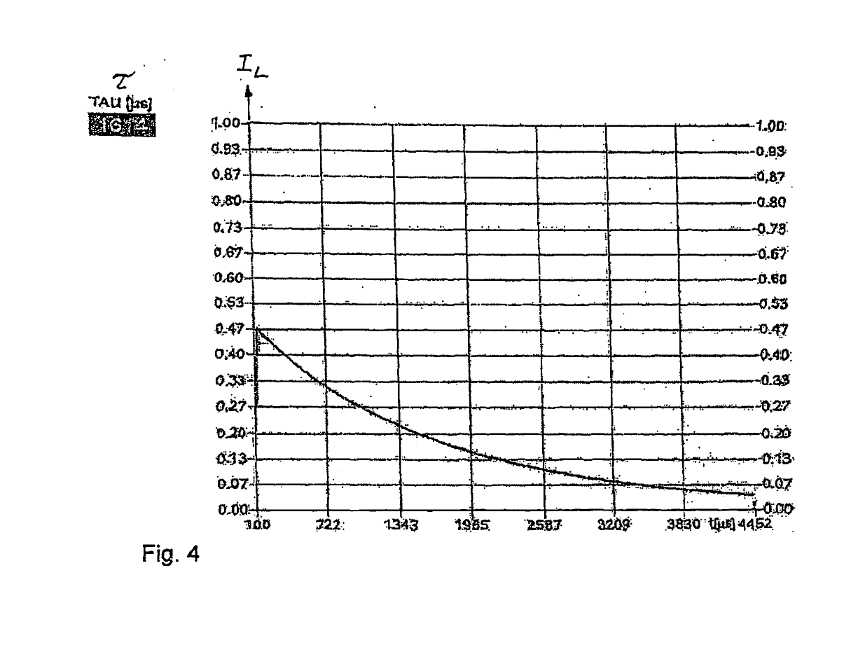

FIG. 4 a time resolved detected luminescence intensity course prior to a treatment by an irradiation,

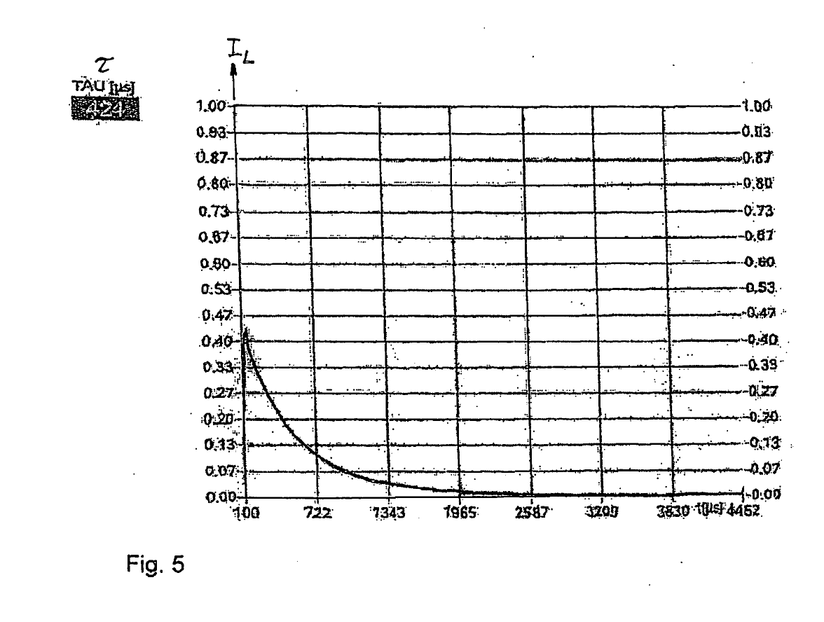

FIG. 5 a time resolved detected luminescence intensity course after a treatment by irradiation.

The method for testing of objects 1 of energy entering treatments 14 according to the present invention exhibits the following steps with consideration of the device 10 according to FIG. 3:
Bonding of at least one chemical compound 3 optically capable of luminescence, to an indicator element 6 for testing for at least one energy entering treatment 14, wherein the luminescence property of the chemical compound 3 is changeable,
Coordinating at least one indicator element 6 to the object 1, wherein the indicator element 6 and the object 1 are simultaneously subjected to the same conditions of the respective energy entering treatment 14,
Changing a luminescence property of the chemical compound 3, wherein the reachable level of the change of the luminescence property depends on the energy entering treatment 14,
Luminescence triggering irradiation 13 of the chemical compound 3 with an electromagnetic radiation for exciting of luminescence during the energy entering treatment 14 or following to the energy entering treatment 14 for detecting the energy entering continuous/instantaneous treatment 14 or the treatment 14 performed energy entering,
Time resolved and/or spectral resolved detection of the emitted electromagnetic radiation 12 as a consequence of luminescence during the irradiation 13 or after switching off of the irradiation 13 with a detector 5,
Making ready of time resolved and/or spectral resolved detection signals 17 from the detector 5 to at least one evaluation unit 9, wherein there occur
a comparison between the detection signals 17 and the reference signals 18 out of at least one previously determined or pre-given reference value and/or at least one reference wavelength or out of a reference wavelength spectrum and
a provable determination of an energy entering performed treatment 14 on the object 1, as well as
At least one display in a display unit 15 on the basis of the changed luminescence property achieved in the chemical compound 3 about the presence of at least one energy entry in the object 1.

At least one chemical compound 3 can be used, which exhibits a reversible change or an irreversible change of the luminescence property, for the detection during the energy entering treatment 14.

At least one chemical compound 3 can be employed, which compound 3 preferably exhibits an irreversible change of the luminescence property, for a detection after termination of the energy entering treatment 14, wherein the irreversibility of the change of at least one luminescence property of the employed chemical compound 3 represents a time stable change of the luminescence property of the chemical compound 3, wherein the time stable change is either a shortening or lengthening of the luminescence lifetime τ, a change in the luminescence spectrum or an increase or decrease of the luminescence intensity I_{L}, wherein selectively the time stable change is tested based on the irreversibility at each time after the energy entering treatment 14.

A luminescence lifetime τ belonging to the chemical compound 3 and/or an associated luminescence intensity can be determined after the energy entering treatment at a pre-givable time and can be compared with at least one reference value.

In case of a time resolved detection the irradiation 13 can be performed as pulses for exciting of luminescence.

The presence or absence of at least one wavelength in the wavelength spectrum of the radiation 12 can be detected as a consequence of luminescence.

Also several chemical compounds 3 can be employed at one or at several indicator element(s) 6, which chemical compounds can retain their stable impressed luminescence property upon reaching of also different energy entries.

The energy entering treatment 14 of medical implants, prostheses, medical apparatus and instruments with electrode irradiation can lead to their sterilization.

Furthermore the energy entering treatment 14 and the detection of time resolved and/or spectral resolved luminescence detection signals 17 can be performed at objects 1 received in hermetically closed containers and the indicator element(s).

The chemical compound(s) 3 can be employed as a powder with an average particle size in the region from 0.001 μm up to 30 μm.

Furthermore, the chemical compound(s) can be received in a separate container 2 in the equipping of an indicator element 6, together with a matrix material be printed on a substrate or on the container wall or immediately attached at the respective object 1 or can be embedded in a polymeric working material or in the work material of the object 1. At least one indicator element 6 formed in this manner can thereby be employed.

The device 10 performing the method for testing of energy entering treatments 14 on objects 1 is illustrated in FIG. 3, wherein the treatments 14 are performed at least in the device 8 for performing an energy entering treatment 14, and wherein the device 10 comprises
At least an indicator element 6 coordinated to an object 1, wherein the indicator element 6 is formed with at least one chemical compound 3, which is capable of optical luminescence and where the luminescence property of the chemical compound is reversible or irreversible changeable, wherein the indicator element 6 is coordinated to the object 1 such that the chemical compound(s) 3 is/are received in a separate container or together with a matrix material are pressed or printed on a wall of the container 2 or the chemical compound(s) 3 are immediately attached at the respective object 1 as indicator elements 6 or are embedded in a polymeric material, the material of the object 1, and
at least one radiation source 4 with which an electromagnetic radiation 13 is emitted onto the indicator element 6 for exciting of luminescence in the chemical compound 3,
At least one optical detector 5 which is formed for the time resolved and/or spectral resolved detection out of a luminescence radiation 12 emitted by the chemical compound 3, and
At least one evaluation unit 9 for the detective determination of at least one treatment performed energy entering onto the object 1.

A control unit 11 and the evaluation unit 9 can be received in the device 10, which control unit 11 and evaluation unit 9 control the irradiation 13 leading to the excitation of luminescence and which evaluate the measurement signals 17 captured with the optical detector 5.

The radiation source 4 and the optical detector can be received in a common apparatus or housing 7.

At least one display for the display unit 15 of a detection result as well as an interface for a data exchange can be present such that a manually led and actuated device 10 is present, which device 10 performs automatically the detection guiding for a running/momentary energy entering treatment 14 and which immediately indicates the detection result.

At least one memory storage 16 can be furnished for the reference values 18 or for the reference wavelengths, which are specific in particular for not influenced chemical compounds 3, which memory storage 16 is integrated into the evaluation unit 9 and the control unit 11.

The luminescence radiation 12 employed for the excitation with electromagnetic radiation 13 as well as the luminescence radiation 12 emitted by an indicator element 6 can be led and guided through deformable light-wave conductors.

As can be recognized for FIG. 1, an object 1 is equipped with an indicator element 6 and the chemical compound 3 in the process I (binding) can be cleaned in a process II (cleaning), then in the process III (enclosure) be closed off hermetically from the ambient in a container 2.

An irradiation 14 of the object 1 in the process IV (treatment) for sterilization is performed through the closed container 2 with electrode irradiation, which container 2 is advanced thereby in the process V (sterilization). An indicator element 6 is furnished within the container 2 already during the irradiation 14. The indicator element 6 comprises a placed carrier, wherein the luminescence chemical compound 3 is printed on the carrier with a dispersion lacquer as a matrix, such as a printing ink. The printing of the indicator element 6 can also be performed on the inner wall of the container 2. The container 2 can be a blister pack known in principle of which one part is formed of an optically transparent polymer foil and another part of paper or aluminum coated with a polymer.

Doped zinc sulfate, doped calcium sulfite, doped aluminum gallate, doped calcium tungstate, doped aluminate chromate, doped rare earth compounds, such as for example rare earth fluorides, or doped oxi-sulfide, or doped metal oxides can be employed as chemical compounds 3.

FIG. 2 illustrates a process IV, where the irradiation 14 can be performed for sterilization. In order to reach the complete surface of objects 1 to be sterilized, the irradiation 14 is performed of two oppositely disposed electron beam sources 8 in this example.

This effect however can also be obtained with a single electron beam source 8 with simultaneous motion, for example rotation of the object 1 with the container 2. The irradiation 14 with electrons can for example be performed with an electron energy of for example 200 keV over a time period of 100 milliseconds such that a dose of the irradiation 14 of 30 kGy is disposed, which was sufficient for the sterilization.

If an electron energy of 200 keV is not reached with the irradiation, then not only the sterilization is performed in a sufficient measure. Also no change of the luminescence properties of the chemical compound 3 at the indicator element 6 occurs. When the entered electron energy is sufficient, however the radiation dose is too small, then the crystal lattice of the respective chemical compound(s) is not or only to a small part changed in volume, which effects an insufficient change in the luminescence properties. A mixing/overlap of the origin of luminescence properties prior to the irradiation 14 with those after the irradiation can occur. The original luminescence lifetimes τ can superpose with the luminescence lifetimes τ changed by the irradiation 14 and this can then also be detected. In this way it can be possible also to detect the in fact entered radiation dose. The part of or the amount of chemical compound 3 present in the indicator element 6, which chemical compound can change its luminescence properties as a consequence of the irradiation 14, can be selected according to the respective irradiation dose.

An electromagnetic radiation 13, from a radiation source 4, for example a laser diode or an LED, with a wavelength from 900 nm up to 1000 nm, a power smaller than 1 W, preferably smaller than 100 mW with a pulse length smaller than 5 ms, which can also be smaller than 1 ms, is directed onto the indicator element 6 in the container 2 and fluorescence or luminescence is excited for detecting the actually reached sterility. The thereby emitted luminescence radiation has a wavelength in the wavelength region from 1000 nm to 1300 nm. This capturing is performed at times, where the radiation source 4 did not emit radiation for excitation. The radiation source 4 and the detector 5 were operated with corresponding triggers.

Between the radiation source 4 emitting the radiation 13 stimulating the luminescence and the detector 5 there can be disposed a trigger 20 as a control unit, wherein the trigger causes the release of a pulse of the electromagnetic radiation 13 onto the indicator element 6, and wherein the trigger 20 signals the capturing of the luminescence radiation 12 with the optical detector 5 with respect to the release to the detector 5 and the two processes show commonality.

The diagram shown in FIG. 3a on the right hand side presents the decay behavior 19 of the luminescence intensity I_{L} depending on the time t after the excitation radiation 13 with an infrared (IR) pulse. The value typical for the lifetime τ lies with this indicator element 6, for the chemical compound not influenced by the treatment with electron radiation as for example at 1614 μs. The chemical compound 3 is in this case a rare earth fluoride (FIG. 4).

A lifetime τ of 424 μs (FIG. 5) was determined in this example however after the sterilization with the electron irradiation. A significant difference of the lifetime τ could be captured by the irreversible change of the luminescence properties and a safe detection for a successfully performed sterilization of the object 1 received hermetically protected in the container 2 could be established without that the object 1 or the container 2 would be destroyed.

The radiation source 4 and the detector 5 are placed in a common housing 7 in FIG. 3 and this way can form a hand measuring instrument.

FIG. 4 and FIG. 5 show respective time resolved detected luminescence intensity courses prior to a treatment and after treatment with a radiation. It becomes clear that the luminescence lifetime τ prior to such irradiation was determined to a value 1614 μs and after irradiation was determined to a value 424 μs. This represents a significant difference, which is sufficient for a detection of a performed treatment 14.

### LIST OF REFERENCE CHARACTERS

1 object
2 container
3 chemical compound
4 radiation source
5 detector
6 indicator element
7 housing
8 apparatus for performing an energy entering treatment
9 evaluation unit
10 device for testing
11 control unit
12 luminescence radiation
13 excitation radiation
14 energy entering treatment
15 display unit
16 memory storage
17 conductor for detection signals
18 reference signals
19 decay behavior
20 trigger
τ luminescence lifetime
I_{L} luminescence intensity
t time
I process—bonding
II process—cleaning
II process—enclosure
IV process—treatment
V process—sterilization

1. Method for testing of energy entering treatments (14) on objects (1) comprising the following steps
bonding of at least one chemical compound (3) capable of optical luminescence, on at least one indicator element (6) for testing the treatments (14), wherein at least one luminescence property of the chemical compound (3) is changeable,
coordinating of at least one indicator element (6) to the object (1), wherein the indicator element (6) and the object (1) are simultaneously subjected to the same conditions of the energy entering treatment (14),
changing of the luminescence property of the chemical compound (3), wherein the level of the change of the luminescence property depends on the energy entering treatment (14),
irradiation (13) of the chemical compound (3) with an electromagnetic radiation directed toward the indicator element (6) for exciting of luminescence during the energy entering treatment or following to the energy entering treatment (14) for detection of energy entering momentary treatment (14) or energy entering performed treatment (14),
time resolved and/or spectrum resolved detection of the electromagnetic radiation (12) by the indicator element (6) emitted in consequence of luminescence of the chemical compound during the luminescence triggering irradiation (13) or after the switching off of the luminescence triggering radiation (13),
ready making of time resolved and/or spectrum resolved detection signals from a detector (5) to at least one evaluation unit (9), wherein there occur
a comparison between the detection signals (17) at the reference signals (18) stored in a memory storage (16) from at least one prior determined reference value and/or at least one reference wavelength or from a reference wavelength spectrum, and
a detection determination of treatment(s) (14) performed energy entering on the object (1), and
at least one display in a display unit (15) on the basis of the accomplished changed luminescence property of the chemical compound (3) relating to the presence of at least one energy entry (14) into the object (1).

2. Method according to claim 1,
characterized in that,
at least a chemical compound (3) is employed for a detection during the energy entering treatment (14), wherein the chemical compound exhibits a reversible change of the luminescence property or an irreversible change of the luminescence property.

3. Method according to claim 1,
characterized in that,
a chemical compound (3) is employed for a detection after finishing the energy entering treatment (14), wherein the chemical compound (3) exhibits an irreversible change of the luminescence property, wherein the irreversibility of the change of at least one luminescence property of the chemical compound (3) presents a time stable change of the luminescence property of the chemical compound (3) wherein the time stable change is either a shortening or a lengthening of the luminescence lifetime τ, a change in the luminescence spectrum or an increase or decrease of the luminescence intensity I_{L}, wherein as selected based on the irreversibility the time stable change is tested at any time after the energy entering treatment(s) (14).

4. Method according to claim 1,
characterized in that,
the luminescence lifetime τ belonging to the chemical compound (3) and/or a luminescence intensity I_{L} belonging to the chemical compound (3) is determined at a pre-given time and is compared with at least one reference value (18).

5. Method according to claim 1,
characterized in that,
upon a time resolved detection, the radiation (13) directed onto the indicator element (6) for exciting of luminescence of the chemical compound (3) is performed as pulses.

6. Method according to claim 1,
characterized in that,
the presence or absence of at least one wavelength in the wavelength spectrum is detected in the emitted radiation (12) based on luminescence.

7. Method according to claim 1,
characterized in that,
several chemical compounds (3) are employed at one or several indicator element(s) (6), wherein the indicator element(s) change their luminescence properties upon reaching different energy entries (14).

8. Method according to claim 1,
characterized in that,
the energy entering treatments) (14) is/are performed with an electron irradiation of medical implants, prostheses, medical apparatus and instruments for their sterilization.

9. Method according to claim 1,
characterized in that,
the energy entering treatment (14) and the detection of time resolved and/or spectrum resolved luminescence detection signals (17) in the detector (5) is performed at objects (1) received in hermetically closed containers (2) and the coordinated indicator element(s) (6).

10. Method according to claim 2,
characterized in that,
the chemical compound(s) are employed in powder form with an average particle size in the region of 0.001 μm to 30 μm.

11. Method according to claim 2,
characterized in that,
in a formation of an indicator element (6), the chemical compound(s) are received in a separate container (2), are together with a matrix material printed on a substrate or on the wall of the container or are attached immediately at the respective object (1) or are embedded in a polymeric working material or in the work material of the object (1) and thereby at least the so formed indicator element (6) is employed.

12. Method according to claim 2,
characterized in that,
doped zinc sulfate, doped calcium sulfite, doped aluminum gallate, doped calcium tungstate, doped aluminate chromate, doped rare earth compounds, such as for example rare earth fluorides, or doped oxi-sulfides or doped metal oxides are employed as chemical compounds (3).

13. Apparatus (10) for testing of energy entering treatment (14) directed to objects (1), wherein the treatments (14) are performed directed by a device (8) for performing of an energy entering treatment (14) with use of at least a part of the steps of the method according to claim 1,
characterized in that,
the device (10) comprises
at least an indicator element (6) coordinated to an object (1), wherein the indicator element (6) is formed with at least a chemical compound (3), which is capable of optical luminescence and where the luminescence property of the chemical compound (3) is changeable,
with at least one radiation source (4) with an electromagnetic radiation (13), wherein the radiation (13) is emitted depending on the need for exciting of luminescence onto the indicator element (6) with the chemical compound (3), wherein the chemical compound (3) exhibits the treatment caused obtained change of the luminescence property, and
at least one optical detector (5), which is formed for the time resolved and/or spectrum resolved detection of luminescence radiation (12) emitted by the chemical compound (3), and
at least one evaluation unit (9) receiving the detected signals of the detector (5) for detecting determination of at least one treatment (14) performed energy entering on the object (1), wherein the treatment (14) is based on the obtained changed luminescence property of the chemical compound (3).

14. Apparatus according to claim 13,
characterized in that,
the indicator element (6) is coordinated to the object (1) such that the chemical compound(s) (3) are received in a separate container or are printed with a matrix material on a substrate or onto the wall of a container or are attached immediately at the respective object (1) or are embedded in a polymeric material, which is the material of the object (1).

15. Apparatus according to claim 13,
at least a chemical compound (3) is integrated into the indicator element (6), wherein the luminescence property of the chemical compound (3) depending on the application during or after the treatment (14) is reversible changeable or irreversible changeable.

16. Apparatus according to claim 13,
characterized in that,
the radiation source (4) emitting the radiation (13) characterized as excitation radiation and the optical detector (5) are received in a common apparatus or in a housing (7).

17. Apparatus according to claim 13,
characterized in that,
a control unit (11) and the evaluation unit (9) are integrated into the device (10), which control unit (11) and evaluation unit (9) control the radiation (13) leading to the excitation of the luminescence and evaluate the measurement signals (17) captured by the optical detector (5).

18. Apparatus according to claim 13,
characterized in that,
a trigger (20) operating as a control unit is disposed between the radiation source (4) emitting the radiation exciting the luminescence and the detector (5), which trigger (20) initiates the release of a pulse of the electromagnetic radiation (13) onto the indicator element (6) and which signals the capturing of the following luminescence radiation (12) to the detector (5) by way of the optical detector (5) with respect to the triggering.

19. Apparatus according to claim 13,
characterized in that,
at least a display for the indicator (15) of the detection result as well as an interface for data exchange is present, such that a manually led and actuated device (10) is present, which device (10) performed automatically the detection operation for a momentary or performed energy entering treatment (14) and which indicates immediately the detection result.

20. Apparatus according to claim 13, characterized in that,
at least one memory storage (16) is furnished for reference values (18) or for reference wavelengths, which are specific in particular for not influenced chemical compounds (3), wherein the memory storage (16) is integrated in the evaluation unit (9) and in the control unit (11).

21. Apparatus according to claim 13,
characterized in that,
the detection operation refers to a momentary or performed energy entering treatment (14) and the detection result refers to the sterilization of medical implants, prostheses, medical apparatus and instruments.

22. Apparatus according to claim 13, characterized in that,
the electromagnetic radiation (13) employed for the excitation of the luminescence as well as also the luminescence radiation (12) emitted by an indicator element (6) are led over flexible deformable lightwave conductors.
